(19) United States
(12) Patent Application Publication
(10) Pub. No.: US 2025/0020663 A1
(43) Pub. Date: Jan. 16, 2025

(54) METHOD FOR THE MANAGEMENT OF ANTIBIOTIC ADMINISTRATION

(71) Applicant: ABIONIC SA, Epalinges (CH)

(72) Inventors: Nicolas DURAND, Blonay (CH); Iwan MÄRKI, Yverdon-les-Bains (CH); Romy BENNINGA, Lausanne (CH); Patrick VAN DEN BOGAARD, Morges (CH); François VENTURA, La Croix sur Lutry (CH)

(21) Appl. No.: 18/708,393

(22) PCT Filed: Nov. 25, 2021
(86) PCT No.: PCT/IB2021/060988

§ 371 Date: May 8, 2024

(51) Int. Cl. G01N 33/68

(52) U.S. Cl. CPC G01N 33/6893; G01N 2333/705; G01N 2800/52

(57) ABSTRACT

The present invention relates to a method for determining the criteria from which an antibiotic administration to a human subject or an animal subject is to be initiated, modified, withhold or stopped. The method may comprise the steps of determining a first level of pancreatic stone protein/regenerative protein (PSP/reg) in a sample of said human subject or said animal subject and comparing the level of PSP/reg with a predetermined threshold. A level of PSP/reg above the predetermined threshold is an indication that the antibiotic administration is to be initiated or is to be modified. A level of PSP/reg below to predetermined threshold is an indication that the antibiotic administration is to be withheld or is to be stopped.

### FIELD OF INVENTION

The present disclosure relates to a method for managing antibiotic administration, in particular for guiding physicians in the decision to start or modify antibiotic to be administered.

### BACKGROUND OF THE INVENTION

Antibiotics are medicines that are used to prevent and treat bacterial infections, which have revolutionized healthcare since the 1930s and have been paramount in saving millions of lives. However, a systematic overuse and misuse of antibiotics is leading to a concerning increase in the number of antibiotic resistant bacteria, which are becoming harder to treat and causing more severe and fatal infections. International and national healthcare organizations such as the World Health Organization (WHO) and the Centers for Disease Control and Prevention (CDC) have identified antibiotic resistance as one of the biggest threats to global health today. The over-prescription of antibiotics by healthcare workers is a key contributing factor leading to resistance, with a direct relationship between antibiotic consumption and the emergence and dissemination of resistant bacteria strains (Nature. 2013: 495 (7440): 141). Studies have shown that antibiotic therapy is incorrectly prescribed in as many as 60% of patients receiving hospital care, and is likely to be even higher outside this environment (Luyt et al., Critical Care, 2014). Aside from contributing to resistance, inappropriate antibiotic use may lead to increased mortality and severe disease, increased healthcare costs, increased lengths of stay in hospital and the need for more complex treatments leading to a higher risk for adverse effects and unwanted drug interactions.

Current antibiotic consumption is on an upward trend due to an increasing ageing population, more frequent surgical interventions requiring prophylactic treatment and an increasing incidence of sepsis (Klein et al., PNAS, 2018). Clinicians use a combination of clinical signs and symptoms to evaluate whether a patient requires antibiotics or not. However, the non-specific clinical picture of infection and sepsis makes it challenging to confirm or rule out bacterial infection, and antibiotics are often prescribed at the risk of missing an infection. On an individual scale, this might save a life, but on a global scale, this is causing a serious problem.

Microbiological cultures are collected to identify the causative pathogen and eventually guide the use of appropriate antibiotics, but these can take between 1-7 days for results to be obtained and cannot be relied on for early treatment. Moreover, the causative pathogen may not be culturable and identification results cannot be obtained. Therefore, a key focus of antibiotic stewardship efforts lies in the early and rapid identification of patients with sepsis and in particular true bacterial infections and those who will truly benefit from an early administration of broad spectrum, empirical treatment. Data from critically ill adults with septic shock shows that for every hour delay in administering appropriate antibiotics, there is an 8% increase in mortality (Kumar et al., Critical Care Medicine, 2006).

The need for novel biomarker-guided antibiotic strategies is therefore high, and this is further exemplified by the limited value of microbiological cultures in septic patients: in a recent systematic review and meta-analysis of seven studies including a total of 22 655 patients, the proportion of patients with culture-positive sepsis or septic shock was only 40.1% (Yuting et al., Critical Care, 2021). This is signified by the fact that a substantial number of bacterial pathogens are not culturable in a laboratory setting, leading to false negative identification of bacterial infection.

In addition, the accurate identification of patients who do not require antibiotics is just as important to safely reducing unnecessary use, and to allow physicians to focus on identifying the true root cause of disease.

Therefore, it is still of utmost importance to identify early which patients will benefit from immediate antibiotic treatment, and which patients do not require them. Moreover, as the first-line treatment is typically to administer a broad-spectrum antibiotic, it is crucial to closely monitor the patient status and observe when this treatment is failing to eliminate the bacterial infection (e.g., due to antibiotic resistance) and quickly change antibiotic regime. The most recent Surviving Sepsis Campaign (SSC) guidelines from October 2021 emphasize that appropriate antibiotic treatment must be administered within the first hour, highlighting the importance for immediate identification.

Pancreatic stone protein/regenerating protein (PSP/reg) belongs to a family of lectin-binding proteins that reacts much earlier during infection, sepsis, septic shock and organ dysfunction than all other current solutions proposed in the international guidelines (C. S. Singer, et al. JAMA 2016, 315:8:801-810) or medical societies.

A literature review (P. Eggimann, et al., Biomarkers 2019, 13:02:135-145), summarizing 13 PSP/reg studies, was published in 2019 and a promising study (H. J. Klein, et al. Ann Surg. 2020) was published in January 2020 by the Burn center of the University Hospital of Zurich.

Theses numerous publications show that the PSP/reg assay is more specific and sensitive than other biomarkers for the early diagnosis of sepsis, septic shock and multiorgan failure in ICU adults (M. Llewlyn, et al., Crit. Care 2013. 17:2:R60), children (Z. Jiri, et. al., Cytokine 2014, 66:2:106-111), newborn (A. Rass, et al., BioMed. Res. Int 2016, 1-8) and burn (H. J. Klein, et al., World Journal of Surgery 2020, 44:3000-3009) patients. PSP/reg allows the identification of sepsis in post-cardiac surgery patients (H. Klein, et al., PLOS ONE 2015, 10:3:e0120276), PSP/reg predicts outcome in patients with peritonitis in ICU (R. Gukasjan, et al., Crit. Care Med 2013. 41:4:1027-1036), PSP/reg is a biomarker of organ failure in Ventilator-Associated-Pneumonia VAP (L. Boeck, et al., Chest 2011, 140:4:925-932) and a biomarker predicting mortality in adults (Y.-A. Que, et al., Crit. Care 2012, 16:4: R114) and in children (Q. Wu, et al., Med. Sci. Monit 2017. 23:1533-1539).

The values of PSP/reg were determined in the healthy subject in 2015 (E. Schlapbach, et al., BMC Anesthesiol 2015. 15:168). PSP/reg detect early infection and sepsis in traumatized patients (M. Keel, et al., Crit. Care Med 2009, 37:5:1642-1648), in emergency department (L. Garcia de Guardinia-Romualdo, et al., Eur. J. Clin. Invest 2017. 47:4:297-304), in pediatric acute osteomyelitis (C. Cui, et al., Med. Sci. Monit 2017. 23:5211-5217), and in cancer patients with febrile neutropenia (L. Garcia de Guadiana-Romualdo, et al., Clin Chem Lab Med 2019, 57:4:540-548).

The pathophysiological mechanism of PSP/reg is not yet clearly defined (R. Graf, Pancreatology 2020. 20:3:301-304), but studies in rats suggest that that serum PSP/reg in septic patients is predominantly derived through an acute phase response of the pancreas (T. Reding, et al., Oncotarget 2017, 8:30162-30174). In humans, PSP/Reg activates granulocyte neutrophils (M. Keel, et al., Crit. Care Med 2009. 37:51642-1648), which seems to confirm that the PSP/reg might serve as an acute phase protein.

In addition to the benefits of PSP as an emerging biomarker for infection and sepsis, it is well established that a patients' temperature variation acts as a predictor of sepsis, however, in itself has a diagnostic accuracy that is not sufficiently high (Global Sepsis Alliance). Under healthy conditions, temperature may fluctuate diurnally by approximately +/−0.5° C. around a mean of 37.0° C., and according to the Sepsis Alliance, abnormal temperatures outside of basal values are a key indicator of infection and sepsis.

Corporal temperature can be assessed in almost any healthcare setting, from primary care through to specialized intensive care services.

The use of unnecessary antibiotics must be prevented and earlier administration needs to be performed.

Consequently, there is a need to provide a method for determining the criteria from which an antibiotic administration to a human subject or an animal subject is to be initiated, modified, withheld or stopped.

### SUMMARY OF THE INVENTION

The inventors have developed a new method allowing for the rapid, accurate and early guidance of antibiotic administration, using PSP/reg biomarker level determination in subject sample.

It has been surprisingly observed that a PSP/reg level above a predetermined threshold is a strong indication that an antibiotic administration is needed. Alternatively, if antibiotics are already administered, a PSP/reg level above the predetermined threshold is a strong indication that a modification of the antibiotic administration is necessary.

Conversely, it has been surprisingly observed that a PSP/reg level below the predetermined threshold is a strong indication that an antibiotic administration is to be withheld. Alternatively, if antibiotics are already administered, a PSP/reg level below the predetermined threshold is a strong indication that the antibiotic administration is to be stopped.

Readily available clinical sign: corporal temperature was also used in combination with the PSP/Reg biomarker for the rapid, accurate and early guidance of antibiotic administration. Alternatively, if antibiotics are already administered, a modification of the antibiotic administration is necessary

It is one aspect of the present disclosure to provide a method for determining the criteria from which an antibiotic administration to a human subject or an animal subject is to be initiated, modified, withheld or stopped.

Preferably, the method comprises the step of determining a first level of pancreatic stone protein/regenerative protein (PSP/reg) in a sample of said human subject or said animal subject.

Preferably, the method further comprises the step of comparing the level of PSP/reg with a predetermined threshold.

Preferably, a level of PSP/reg above the predetermined threshold is an indication that the antibiotic administration is to be initiated or is to be modified.

Preferably, a level of PSP/reg below to predetermined threshold is an indication that the antibiotic administration is to be withheld or is to be stopped.

In another aspect of the present disclosure, the method comprises the step of determining a second level of PSP/reg in a sample of said human subject or said animal subject after a period of time and comparing the second level of PSP/reg with the first level of PSP/reg, wherein an increase of PSP/reg level between two measurements is an indication that the antibiotic administration is to be initiated or is to be modified and wherein a decrease of PSP/reg level between two measurements is an indication that the antibiotic treatment is to be withheld or is to be stopped.

According to another aspect of the disclosure, the period of time is one day

According to yet another aspect of the disclosure, the increase of PSP/reg level is at least a 50 ng/ml increase and the decrease of PSP/reg level is at least a 50 ng/ml decrease of PSP/reg.

According to still another aspect of the present disclosure, the first level of PSP/reg and/or the second level of PSP/reg are determined in vitro.

According to another aspect of the disclosure, the first level of PSP/reg and/or the second level of PSP/reg are determined at the Point-of-Care and/or in an analysis laboratory.

According to another aspect of the present disclosure, the first level of PSP/reg and/or the second level of PSP/reg are determined by at least one from the list comprising ELISA, RIA, EIA, mass spectrometry, microarray analysis and fluidic assay methods.

According to yet another aspect of the disclosure, the predetermined threshold is 200 ng/ml of PSP/reg.

According to still another aspect, a level of PSP/reg below 200 ng/ml is indicating a low probability of bacterial infection.

According to another aspect of the present disclosure, a level of PSP/reg above 200 ng/ml is indicating a high probability of developing bacterial infection

According to yet another aspect of the disclosure, the method further comprises the step of determining a corporal temperature of the human subject or the animal subject, an increase of the corporal temperature outside a basal range being an indication that the antibiotic administration is to be initiated.

According to still another aspect, the basal range is comprised between 97 F (36.5° C.) to 99 F (37.5° C.) for adults and between 97.9 F (36.6° C.) to 100.4 F (38.0° C.) for babies and children.

According to another aspect of the present disclosure, the sample comprises at least one from the list comprising whole blood, serum, plasma, urine, sputum, cerebrospinal fluid, tear fluid, saliva, sweat, milk, an extract from solid tissue and an extract from fecal matter.

The present invention relates to a method of managing the initiation of antibiotic therapy in patients, wherein the level of pancreatic stone protein/regenerative protein (PSP/reg) is determined in a body fluid sample.

### BRIEF DESCRIPTION OF THE DRAWINGS

The accompanying drawings, which are incorporated herein and constitute part of this specification, illustrate the presently preferred embodiments of the invention, and together with the general description given above and the detailed description given below, serve to explain features of the invention.

FIG. 1A shows a patient case report. Patient was admitted to ICU with severe COVID-19 pneumonia and placed under invasive mechanical ventilation from day 1. This patient received empirical antibiotics during the first 2 days as per hospital protocol. PSP/Reg was monitored in an observational, daily basis. Microbiological broncho aspirate sampling was performed on day 3 with no detection of pathogen. However, the patient did receive unnecessary antibiotics for the first two days. PSP/Reg increased suddenly by 112% between day 5 and day 6, and the patient was observed to have a fever on day 6. Microbiological cultures were then collected on days 7 and 8 (broncho aspirate and broncho lavage respectively), and the results which were obtained on day 9, confirmed the presence of a pathogen, prompting the initiation of antibiotic treatment on day 9.

FIG. 1B shows a similar case report where the method according to the present disclosure was applied. The low PSP/Reg concentration during the first 5 days enables the withholding of unnecessary antibiotics. The sudden increase of PSP/Reg of 112% between day 5 and 6, along with the presence of an abnormal temperature of 38.6° C. triggers the early and now appropriate administration of antibiotics. A decreasing PSP/Reg concentration below 200 ng/ml after the initiation of antibiotics as seen on day 9, indicates the ability to stop unnecessary antibiotics.

FIG. 2 depicts the management of antibiotics administration according to the concentration of PSP/reg and/or corporal temperature.

FIG. 3A shows a box plot of median PSP concentrations with 5th and 95th percentiles and mean shown as a cross. Data points are from adult patients admitted to intensive care unit, grouped according to the days they received antibiotics or not. Mann-Whitney test shows a statistically significant difference between the group of patients who did not require antibiotics (mean 89 ng/ml) and those who did require antibiotics (mean 286 ng/ml) with P-value of 0.0002 (***).

FIG. 3B shows a box plot of median PSP concentrations with 5th and 95th percentiles and mean shown as a cross. A cutoff of 200 ng/ml PSP/reg was apply to the data points and the number of patients receiving unnecessary antibiotics can be reduced from 17 to 9.

### DETAILED DESCRIPTION OF THE INVENTION

The present invention relates to a method for determining the criteria from which an antibiotic administration to a human subject or an animal subject is to be initiated, modified, withheld or stopped.

The method may comprise the step of determining a first level of pancreatic stone protein/regenerative protein (PSP/reg) in a sample of said human subject or said animal subject.

The method may further comprise the step of comparing the level of PSP/reg with a predetermined threshold.

A level of PSP/reg above the predetermined threshold is an indication that the antibiotic administration is to be initiated or is to be modified.

A level of PSP/reg below to predetermined threshold is an indication that the antibiotic administration is to be withheld or is to be stopped.

Antibiotic administration may be withheld until the level of PSP/reg reaches the predetermined threshold which will be an indication that the antibiotic administration is to be initiated or reinitiated.

Antibiotic administration modification may comprise a modification of the type of antibiotic to be administered to a subject or a modification of the antibiotic dosage regiment.

Antibiotic may be selected from the list comprising at least one of penicillins, tetracyclines, cephalosporins, quinolones, lincomycins, macrolides, sulfonamides and glycopeptides.

The method may further comprise the step of determining a second level of PSP/reg in a sample of said human subject or said animal subject after a period of time.

The second level of PSP/reg may be compared with the first level of PSP/reg.

An increase of PSP/reg level between two measurements is an indication that the antibiotic administration is to be initiated or is to be modified.

A decrease of PSP/reg level between two measurements is an indication that the antibiotic treatment is to be withheld or is to be stopped.

The first level of PSP/reg and/or the second level of PSP/reg are determined in vitro for example in analysis laboratory or facility. The human subject or animal subject may not be present or needed during the determination of the first and/or second level of PSP/reg.

The period of time may be one day or 24 hours.

Alternatively, the period of time may be less than one day, for example 6 hours, 12 hours or 18 hours or any suitable time duration.

The increase of PSP/reg level between two measurements may be at least a 50% increase.

The increase of PSP/reg level between two measurements may be at least a 50 ng/ml increase of PSP/reg.

The decrease of PSP/reg level between two measurements may be at least a 50% decrease.

The decrease of PSP/reg level between two measurements may be at least a 50 ng/ml decrease of PSP/reg.

The predetermined threshold may be substantially 200 ng/ml of PSP/reg.

The predetermined threshold may be from 150 ng/ml to 250 ng/ml or from 160 ng/ml to 240 ng/ml or from 170 ng/ml to 230 ng/ml of PSP/reg or from 180 ng/ml to 220 ng/ml of PSP/reg or from 190 ng/ml to 210 ng/ml of PSP/reg.

The predetermined threshold may be substantially 150 ng/ml of PSP/reg.

The predetermined threshold may be substantially 250 ng/ml of PSP/reg.

For example, a threshold of 150 ng/ml of PSP/reg may be used for a human subject who is not in intensive care but for whom an infection is suspected.

Alternatively, a threshold of 200 ng/ml of PSP/reg may be used for a human subject in intensive care without suspicion of infection.

A threshold of 250 ng/ml of PSP/reg may be used for a human subject in intensive care with a suspicion of infection.

A level of PSP/reg above the predetermined threshold is indicating a high probability of developing bacterial infection.

A level of PSP/reg below the predetermined threshold is indicating a low probability of developing a bacterial infection.

A level of PSP/reg above the predetermined threshold is indicating a high probability of developing bacterial infection.

The method of the present disclosure may further comprise the step of determining a corporal temperature of the human subject or the animal subject.

An increase of the corporal temperature outside a basal range may be an indication or a supplemental indication that the antibiotic administration is to be initiated.

The basal range of human subject corporal temperature may be comprised between 97 F (36.5° C.) to 99 F (37.5° C.) for adults and between 97.9 F (36.6° C.) to 100.4 F (38.0° C.) for babies and children.

For animal subject, the basal range may comprise within the same range of human subject basal range or within ranges specific to the specie of the animal subject.

The sample may comprise comprises at least one from the list comprising whole blood, serum, plasma, urine, sputum, cerebrospinal fluid, tear fluid, saliva, sweat, milk, an extract from solid tissue and an extract from fecal matter.

On the one hand the disclosure helps in the determination of early and timely administration of appropriate antibiotics and on the other hand it avoids the routine administration of antibiotics to patients who do not require treatment and guides healthcare professionals in the decision to stop antibiotic therapy.

Very close monitoring of PSP/reg levels allows the adaptation of the regime or selected antibiotic to be administered.

As defined herewith, “PSP/reg” refers to human pancreatic stone protein, also called regenerating gene (REG) I protein or lithostatine or pancreatic thread protein (Gross et al., J. Clin. Invest. 1985, 76:21 15-2126) and can be the isoform alpha (Uniprot sequence number: P05451, also identified herewith as SEQ ID NO:1) or beta (Uniprot sequence number: P48304, also identified herewith as SEQ ID NO:2).

Body fluids useful for determination of PSP/reg levels are e.g. whole blood, serum, plasma, urine, sputum, cerebrospinal fluid, tear fluid, saliva, sweat, milk, or extracts from solid tissue or from fecal matter.

Any known method may be used for the determination of the level of PSP/reg in body fluids. Methods considered are e.g. ELISA, RIA, EIA, mass spectrometry, or microarray analysis and any fluidic assay methods.

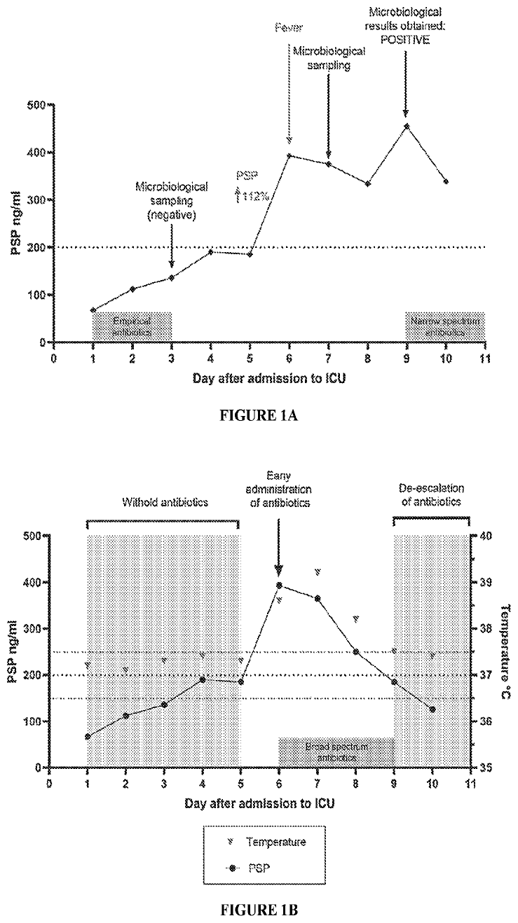

FIG. 1A shows the evolution over 10 days of the level of PSP measured in whole blood for a patient admitted to the ICU with severe COVID-19 pneumonia. The patient was placed under invasive mechanical ventilation from day 1 and received antibiotics for the first two days. On day 3 negative microbiological bronchoaspirate sampling confirmed unnecessary antibiotic therapy during the first two days. Between day 5 and day 6 PSP increased by 112%, and the patient was found to have a fever on day 6. Subsequent positive microbiological bronchoaspirate sampling confirmed bacterial infection and initiated antibiotic therapy on day 9. The presented patient case report clearly illustrates how the PSP-guided antibiotic therapy could have benefited the patient in preventing unnecessary administration of antibiotics as well as in an earlier administration of antibiotics at the onset of infection. In fact, as long as the level of PSP is below a certain threshold antibiotic administration can be withhold and the use of inappropriate antibiotics leading to increased complications and adverse effects can be prevented.

Increased levels of PSP in combination of temperature outside of the normal range may also an indication to initiate administration of antibiotic therapy leading to an earlier treatment starting from day 6 instead of day 9, when microbiological results became available.

FIG. 1B illustrates a patient journey similar to FIG. 1A but when applying the here presented method of antibiotic management based on the measurement of PSP and temperature levels. A low level of PSP during the first 5 days allows to withhold unnecessary antibiotic treatment reducing complications, the risk of adverse effects, the need for more complex treatment as well as the development of antibiotic resistance. The sudden increase of the PSP level by 112% between day 5 and 6, along with the presence of an abnormal temperature can be an indication to trigger the now appropriate administration of antibiotics. This timely administration of antibiotic treatment may help increase better treatment outcomes and reduce lengths of stay in hospitals. PSP levels decreasing to low levels after the initiation of antibiotics as seen on day 9 indicate the phase where de-escalation of antibiotic treatment can take place.

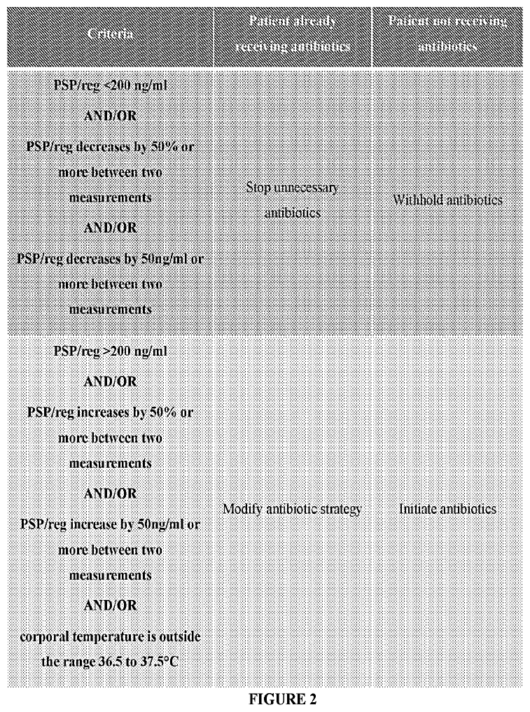

Based on the described patient cases in FIG. 1 a decision table for PSP-guided management of antibiotic therapy is established and shown in FIG. 2. A measured level of PSP that is below 200 ng/ml or a PSP level that has decreased by 50% between two subsequent measurements indicates to the health professional to either stop or to withhold unnecessary antibiotic treatment. PSP levels above 200 ng/ml or an increase of 50% between two subsequent measurements in association with a temperature outside of the normal range may be an indication to either continue or modify an already started antibiotic treatment or to initiate the antibiotic treatment.

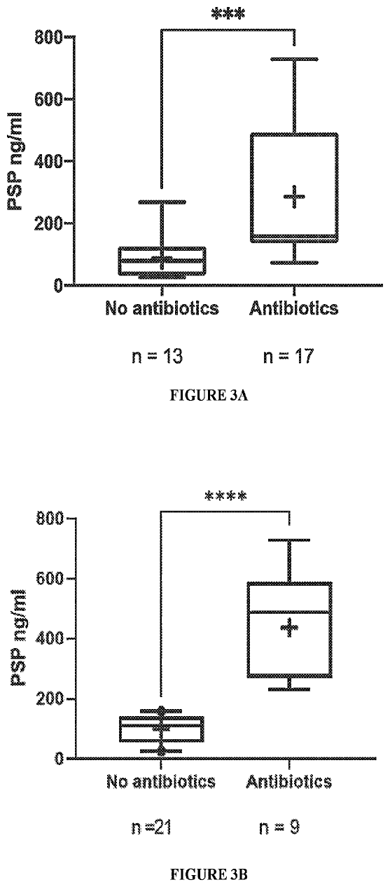

FIG. 3 further underlines the validity of the method of PSP-guided antibiotic management on the initiation or refrain of antibiotic therapy in patients with suspected bacterial infection. Observational PSP/Reg measurements from adult patients receiving ICU treatment were compiled according to whether or not the patient received antibiotics (see FIG. 3A). After applying the cut-off of 200 ng/ml of PSP/Reg, the invertors spontaneously and surprisingly observed a reduction in the number of patients who would require antibiotic treatment from 17 down to 9 (see FIG. 3B).

The invention allows to prevent the use of unnecessary antibiotics and to indicate when an earlier administration of antibiotics is to be administered, potentially leading a reduced risk of antibiotic resistance and better patient treatment outcomes.

While the invention has been disclosed with reference to certain preferred embodiments, numerous modifications, alterations, and changes to the described embodiments, and equivalents thereof, are possible without departing from the sphere and scope of the invention. Accordingly, it is intended that the invention is not limited to the described embodiments and be given the broadest reasonable interpretation in accordance with the language of the appended claims.

1. A method for determining the criteria from which an antibiotic administration to a human subject or an animal subject is to be initiated, modified, withheld or stopped, the method comprising the step of:
determining a first level of pancreatic stone protein/regenerative protein (PSP/reg) in a sample of said human subject or said animal subject;
comparing the level of PSP/reg with a predetermined threshold;
wherein a level of PSP/reg above the predetermined threshold is an indication that the antibiotic administration is to be initiated or is to be modified;
wherein a level of PSP/reg below to predetermined threshold is an indication that the antibiotic administration is to be withheld or is to be stopped.

2. The method according to claim 1, further comprising the steps of:
determining a second level of PSP/reg in a sample of said human subject or said animal subject after a period of time and comparing the second level of PSP/reg with the first level of PSP/reg;
wherein an increase of PSP/reg level between two measurements is an indication that the antibiotic administration is to be initiated or is to be modified;
wherein a decrease of PSP/reg level between two measurements is an indication that the antibiotic treatment is to be withheld or is to be stopped.

3. The method according to claim 2, wherein the period of time is one day.

4. The method according to claim 2, wherein the increase of PSP/reg level is at least a 50 ng/ml increase and the decrease of PSP/reg level is at least a 50 ng/ml decrease of PSP/reg.

5. The method according to claim 1, wherein the first level of PSP/reg and/or the second level of PSP/reg are determined in vitro.

6. The method according to claim 5, wherein the first level of PSP/reg and/or the second level of PSP/reg are determined at Point-of-Care or in an analysis laboratory.

7. The method according to claim 5 or 6, wherein the first level of PSP/reg and/or the second level of PSP/reg are determined by at least one from the list comprising ELISA, RIA, EIA, mass spectrometry, microarray analysis and fluidic assay methods.

8. The method according to claim 1, wherein the predetermined threshold is comprised between 150 ng/ml of PSP/reg to 250 ng/ml of PSP/reg.

9. The method according to claim 1, wherein the predetermined threshold is 200 ng/ml of PSP/reg.

10. The method according to claim 9, wherein a level of PSP/reg below 200 ng/ml is indicating a low probability of developing bacterial infection.

11. The method according to claim 9, wherein a level of PSP/reg above 200 ng/ml is indicating a high probability of developing bacterial infection.

12. The method according to claim 1, further comprising the step of:
determining a corporal temperature of the human subject or the animal subject;
wherein an increase of the corporal temperature outside a basal range is an indication that the antibiotic administration is to be initiated.

13. The method according to claim 12, wherein the basal range is comprised between 97 F (36.5° C.) to 99 F (37.5° C.) for adults and between 97.9 F (36.6° C.) to 100.4 F (38.0° C.) for babies and children.

14. The method according to claim 1, wherein the sample comprises at least one from the list comprising whole blood, serum, plasma, urine, sputum, cerebrospinal fluid, tear fluid, saliva, sweat, milk, an extract from solid tissue and an extract from fecal matter.
